(19) United States
(12) Patent Application Publication
(10) Pub. No.: US 2016/0082002 A1
(43) Pub. Date: Mar. 24, 2016

(54) METHOD OF REGULATING CARDIAC ARRHYTHMIAS

(71) Applicant: James J. Eberl, Hilton Head Island, SC (US)

(72) Inventor: James J. Eberl, Hilton Head Island, SC (US)

(21) Appl. No.: 14/494,636

(22) Filed: Sep. 24, 2014

(51) Int. Cl. A61K 31/4709

(52) U.S. Cl. CPC A61K 31/4709

(57) ABSTRACT

A method of treating cardiac arrhythmia. The method comprises oral ingestion of liquid comprising quinine, or the stereoisomer quinidine. The liquid, which may be a beverage, is consumed in a volume that provides quinine equivalent to consumption of not less than 2 milligrams and not more than 80 milligrams per day. This dosage has been found to be effective in treating cardiac arrhythmia and inhibiting physiologically damaging atrial fibrillation, and the complications thereof.

### FIELD OF THE INVENTION

This invention relates to cardiac arrhythmias and methods of treatment.

### BACKGROUND OF THE INVENTION

Cardiac arrhythmias may be associated with severe complications. With atrial fibrillation, the chaotic rhythm may cause blood to pool in the heart's upper chambers (atria) and form clots. If a blood clot forms, it may dislodge and travel to the brain, where the clot blocks blood flow, and causes a stroke. Atrial fibrillation, if not controlled, may weaken the heart and lead to heart failure.

It is known that quinine can have an effect on heart palpitations, such as atrial fibrillation. In the past, quinine has been prescribed in dosages of 1 gram per day or higher to treat atrial fibrillation. The daily therapeutic dose of quinine for medical uses has been in the range of 500-1000 milligrams (mg) per day.

However, quinine treatments in higher doses are linked to severe, and even fatal, medical complications. Mild overdoses may result in flushed or sweaty skin, ringing in the ears, confusion, blurred vision, abdominal pain and headache. Severe overdoses may result in death. In 1995, the US Food and Drug Administration banned quinine for medical uses other than treatment of malaria.

In the United States, the Food and Drug Administration (FDA) limits quinine content in tonic water for consumption as a beverage to 83 parts per million (83 mg per liter if calculated by mass). 85 mg per liter is the maximum concentration of quinine in tonic water permitted in Germany.

Anticlotting drugs are used to treat atrial fibrillation. However, these drugs are not always effective. Anticlotting drugs are linked to increased bleeding risk, with a risk of major hemorrhaging present in some cases.

### SUMMARY OF THE INVENTION

The invention is a method of treating cardiac arrhythmia. Treatment according to the method may be prophylactic, to prevent the occurrence of cardiac arrhythmia, or treatment may restore and/or maintain normal heart rhythm for a person who is experiencing, or has experienced, arrhythmia.

The method of the invention comprises oral ingestion of liquid comprising quinine, or the stereoisomer quinidine. The liquid, which may be a beverage, is consumed in a volume that provides quinine equivalent to consumption of not less than 2 milligrams (mg.) and not more than 80 milligrams per day. More preferably, oral ingestion of the liquid beverage occurs at a lower dosage twice a day, with each dose taken 8 to 16 hours apart. These dosages have been found to be effective in treating cardiac arrhythmia and inhibiting physiologically damaging atrial fibrillation, and the complications thereof.

### DESCRIPTION OF PREFERRED EMBODIMENTS

The present invention is a method of treating cardiac arrhythmia by the oral ingestion of low dosages of quinine or its stereoisomer quinidine. The inventor has determined that consuming low dosages of tonic water containing quinine on a daily basis substantially eliminates cardiac arrhythmia. For the purposes of this invention, quinidine is a substitution for quinine. Treatment according to the method may be prophylactic, to prevent the occurrence of cardiac arrhythmia, or treatment may be used to restore and/or maintain normal heart rhythm for a person who is experiencing, or has experienced, arrhythmia.

According to the invention, quinine or quinidine are orally ingested in a volume of 2 mg. to 80 mg. per day. Lower dosages than 2 mg. per day are not proven to be effective, and the dosages according to the invention are efficacious while avoiding the complications of substantially higher dosages of quinine and quinidine that have been observed.

More preferably, 4 mg. to 40 mg. of quinine or quinidine are orally ingested twice per day, with the second dose ingested about 6 to 12 hours after the first dose. The half-life of orally ingested quinine/quinidine in the body is about 6-8 hours. It is preferred that no more than 40 mg. of quinine or quinine is ingested in any 8 hour period, although the invention is believed to be effective for materially reducing or eliminating atrial fibrillation if 10-20 mg. is ingested once per day.

Grocery store tonic water is sold as a beverage under brands such as Schweppes, Canada Dry, and private supermarket labels. Grocery store tonic water has a concentration in the United States of 70-83 mg. of quinine per liter of tonic water, with about 80 mg. per liter being common. Quinine is added as a flavoring agent to the tonic water by the manufacturer. The tonic water comprises natural or artificial sweeteners and flavorings added to carbonated water. Quinine is dissolved in the tonic water in the form of a salt. The salt is commonly quinine hydrochloride.

Oral ingestion of a quinine beverage is preferred. For grocery store tonic water, a volume of not less than about 60 milliliters and not more than about one liter per day is preferred, so as to achieve the preferred range of 4 mg. to 80 mg. of quinine/quinidine per day for common concentrations of quinine/quinidine in grocery store tonic water. The beverage, which may be tonic water, may have a concentration of quinine of not less than 70 milligrams and not more than 85 milligrams per liter of the beverage.

In a preferred embodiment, tonic water of the type that is available at grocery stores for use as a beverage is consumed in the morning and in the evening in a volume of about 50 milliliters (ml) per dose. For two subjects who consumed such dosages over a period of about seven (7) years, only two occurrences of atrial fibrillation were observed, and neither was in excess of two minutes in length of time. One subject is a male who is more than 80 years of age with a prior history of heart problems and atrial fibrillation, who was monitored substantially continuously by means of a pacemaker that records heart activity. The other subject is a female over 60 years of age with no history of heart problems or atrial fibrillation. Without being bound by theory, the inventor believes that ingestion of quinine according to the invention eliminates the occurrence of physiologically damaging atrial fibrillation. It is also believed that the invention prevents premature atrial complexes from forming.

Tonic syrups are also available. Water, or carbonated water such as soda water, may be mixed with the tonic syrup. The syrup or water made from the syrup may be used according to the invention. The amount quinine consumed according to the invention should be 2 mg to 80 mg. per day, and preferably 4 mg. to 40 mg. per day as described herein. Quinine in a solid form may be dissolved in water or carbonated water to form the liquid for consumption according to the method of the invention.

1. A method of treating cardiac arrhythmia, comprising the steps of oral ingestion of a therapeutic agent, wherein the therapeutic agent consists of quinine or quinidine in a dose of not less than 2 milligrams per day and not more than 80 milligrams per day.

2. A method of treating cardiac arrhythmia, as described in claim 1, wherein the therapeutic agent consists of quinine or quinidine in a dose of not less than 4 milligrams per day and not more than 40 milligrams per day.

3. A method of treating cardiac arrhythmia as described in claim 1, wherein the therapeutic agent is orally ingested as quinine or quinidine twice per day in a cumulative dosage of not less than 2 milligrams per day and not more than 80 milligrams per day.

4. A method of treating cardiac arrhythmia as described in claim 2, wherein the therapeutic agent is orally ingested as quinine or quinidine twice per day in a cumulative dosage of not less than 4 milligrams per day and not more than 40 milligrams per day.

5. A method of treating cardiac arrhythmia as described in claim 1, wherein the therapeutic agent is orally ingested as quinine or quinidine in two doses per day in a cumulative dosage of not less than 2 milligrams per day and not more than 80 milligrams per day, and wherein an additional dose is taken not less than 6 hours after the first dose.

6. A method of treating cardiac arrhythmia as described in claim 2, wherein the therapeutic agent is orally ingested as quinine or quinidine in two doses per day in a cumulative dosage of not less than 4 milligrams per day and not more than 40 milligrams per day, and wherein an additional dose is taken not less than 6 hours after the first dose.

7. A method of treating cardiac arrhythmia as described in claim 1, wherein the therapeutic agent consists of quinine or quinidine dissolved in a liquid.

8. A method of treating cardiac arrhythmia as described in claim 2, wherein the therapeutic agent consists of quinine or quinidine dissolved in a liquid.

9. A method of treating cardiac arrhythmia as described in claim 1, wherein the therapeutic agent consists of quinine or quinidine orally ingested as tonic water.

10. A method of treating cardiac arrhythmia as described in claim 2, wherein the therapeutic agent consists of quinine or quinidine orally ingested as tonic water.

11. A method of treating cardiac arrhythmia, comprising the steps of oral ingestion of a therapeutic agent twice per day wherein the therapeutic agent comprises quinine or quinidine in a cumulative dosage of not less than 2 milligrams per day and not more than 80 milligrams per day.

12. A method of treating cardiac arrhythmia as described in claim 11, wherein the therapeutic agent is orally ingested as quinine or quinidine twice per day in a cumulative dosage of not less than 4 milligrams per day and not more than 40 milligrams per day.

13. A method of treating cardiac arrhythmia as described in claim 11, wherein the therapeutic agent is orally ingested as quinine or quinidine in two doses per day in a cumulative dosage of not less than 2 milligrams per day and not more than 80 milligrams per day, and wherein an additional dose is taken not less than 6 hours after the first dose.

14. A method of treating cardiac arrhythmia as described in claim 12, wherein the therapeutic agent is orally ingested as quinine or quinidine in two doses per day in a cumulative dosage of not less than 4 milligrams per day and not more than 40 milligrams per day, and wherein an additional dose is taken not less than 6 hours after the first dose.

15. A method of treating cardiac arrhythmia as described in claim 11, wherein the therapeutic agent comprises quinine or quinidine dissolved in a liquid.

16. A method of treating cardiac arrhythmia as described in claim 12, wherein the therapeutic agent consists of quinine or quinidine dissolved in a liquid.

17. A method of treating cardiac arrhythmia as described in claim 11, wherein the therapeutic agent comprises quinine or quinidine orally ingested as tonic water.

18. A method of treating cardiac arrhythmia as described in claim 12, wherein the therapeutic agent comprises quinine or quinidine orally ingested as tonic water
